(19) United States
(12) Patent Application Publication
(10) Pub. No.: US 2024/0131496 A1
(43) Pub. Date: Apr. 25, 2024

(54) LAYER STRUCTURED MULTIFUNCTIONAL MONOLITH CATALYST FOR ENERGY-EFFICIENT CONVERSION OF CO2 TO DIMETHYL ETHER

(71) Applicant: UT-Battelle, LLC, Oak Ridge, TN (US)

(72) Inventors: Haiying Chen, Oak Ridge, TN (US); Josh A. Pihl, Oak Ridge, TN (US); Todd J. Toops, Oak Ridge, TN (US)

(21) Appl. No.: 18/371,739

(22) Filed: Sep. 21, 2023

Related U.S. Application Data
(60) Provisional application No. 63/419,009, filed on Oct. 25, 2022.

(51) Int. Cl. B01J 23/80; B01J 29/65; B01J 35/00; B01J 35/02; B01J 35/04; B01J 35/10; C07C 41/30

(52) U.S. Cl. CPC B01J 23/80; B01J 29/65; B01J 35/0006; B01J 35/023; B01J 35/04; B01J 35/10; C07C 41/30; C07C 2523/80; C07C 2529/65

(57) ABSTRACT

A layered-structure, multifunctional monolith catalyst is provided. The multifunctional monolith catalyst includes a monolithic substrate. A first layer is coated on a surface of the substrate. The first layer includes a first catalyst. A second layer is formed on top of the first layer. The second layer includes a second catalyst, and the second layer is porous. Layering of the first and second catalysts reduces degradation of one or both of the first and second catalysts, and increases a yield of the reaction catalyzed by the second catalyst. A method of converting carbon dioxide to dimethyl ether using the multifunctional monolith catalyst is also provided.

### CROSS-REFERENCE TO RELATED APPLICATIONS

This application claims the benefit of U.S. Provisional Application No. 63/419,009, filed Oct. 25, 2022, the disclosure of which is incorporated by reference in its entirety.

### STATEMENT REGARDING FEDERALLY SPONSORED RESEARCH AND DEVELOPMENT

This invention was made with government support under Contract No. DE-AC05-00OR22725 awarded by the U.S. Department of Energy. The government has certain rights in the invention.

### FIELD OF THE INVENTION

The present invention relates to catalysts for the conversion of carbon dioxide to other compounds and other applications.

### BACKGROUND OF THE INVENTION

The level of greenhouse gases (primarily water vapor, carbon dioxide, methane, nitrous oxide, and ozone) in the atmosphere is important due to its effect on Earth's average surface temperature. Increased carbon dioxide (CO₂) production and emission in the last 300 years has been one of the largest culprits in the rising level of atmospheric greenhouse gases, which threatens to increase the average surface temperature on Earth. In fact, CO₂ is the largest contributor to global warming and climate change, and by certain estimates its atmospheric concentration has risen to 48% above its pre-industrial level. The global anthropogenic CO₂ emission in 2018 was nearly 3.3×10¹⁰ ton, and currently the atmospheric concentration is nearly 416 ppm. According to reports, by the year 2100 the concentration of CO₂ could reach 570 ppm, resulting in a temperature rise of 1.9° C. and an increase in sea level by 3.8 m.

Transformation of mobility to reach net zero CO₂ emissions is crucial in meeting carbon neutral goals by the middle of this century set by many nations as the transportation sector currently emits roughly 30% of the greenhouse gas emissions that contribute to climate change. While light-duty passenger vehicles and medium-duty trucks can be electrified, certain parts of the transportation sector, such as aviation, maritime, and off-road heavy machinery, will still rely on hydrocarbon (HC) fuels in the foreseeable future to meet the demand for high energy density. Converting CO₂, either from point sources or through direct air capture, to HC fuels with renewable energy and green hydrogen can be a workable path to accelerate the decarbonization of the transportation sector. CO₂ can be hydrogenated to a wide range of fuels or chemicals depending on the catalytic processes chosen. Among these processes, conversion of CO₂ to dimethyl ether (DME) is of great interest. It preserves the highest amount of chemical energy of H₂, and the chemical reactions are more energy efficient because they can be carried out under moderate pressures (˜20-50 bar) and temperatures (˜200-320° C.). DME is a non-toxic clean fuel with physical properties similar to that of liquid petroleum gas (LPG), so it can be transported through established LPG storage and distribution infrastructures. DME also has a high cetane number (>55) and can be used as a substitute for diesel fuel for heavy-duty diesel engines. Additionally, it is a versatile chemical intermediate for production of widely used chemicals and can serve as a H₂ carrier.

The current industrial process for DME production utilizes syngas (a mixture of CO, CO₂, and H₂) as the feedstock and involves two distinct catalytic reaction steps. First, syngas is converted into methanol catalyzed by metal oxides, typically CuO/ZnO/Al₂O₃:

CO+2H₂=CH₃OH ΔH°=−90.6 kJ/mol (R1)

CO₂+3H₂=CH₃OH+H₂O ΔH°=−49.4 kJ/mol (R2)

The produced methanol is subsequently dehydrated in a separate reactor to DME over acid catalysts, such as γ-Al₂O₃ or zeolites:

2CH₃OH=CH₃OCH₃+H₂O ΔH°=−23.4 kJ/mol (R3)

With CO₂ as feedstock, a reverse water gas shift reaction step may also be needed:

CO₂+H₂=CO+H₂O ΔH°=41.2 kJ/mol (R4)

This reduces the water content during the methanol synthesis step improving methanol yield.

The main reason for carrying out these reactions in separate reactors is that each of the catalytic processes can be optimized to achieve the highest efficiency of material conversion. To maximize the overall energy conversion efficiency, these reactors are typically located at a centralized chemical plant. This arrangement requires great capital investment and intense energy supply but does provide economies of scale. However, this arrangement is not compatible with most renewable energy sources as those are generally decentralized and have intermittent availability. To effectively convert renewable energy into storable chemical energy and to reduce capital and operational costs, it is highly desirable to combine different chemical processes into one integrated modular system rather than using separate units in sequential steps. CO₂ can be directly converted to DME in a single reactor but this approach requires a bifunctional catalyst that contains both metal oxides, such as CuO/ZnO/ZrO₂ to catalyze methanol synthesis (R2) and acid components, zeolites, for the dehydration step (R3) to complete the two reactions in tandem. Combining the two catalytic components into one system also creates synergies as the in-situ consumption of methanol in (R3) forces (R2) going forwards to achieve thermodynamic equilibrium, thus improving one-pass CO₂ conversion and energy utilization efficiency.

A considerable amount of research effort has been devoted to the development of suitable bifunctional catalysts for the direct synthesis of DME using CO₂ as the feedstock, but several challenges remain that need to be overcome. Since the CO₂ hydrogenation reaction (R2) is exothermic and leads to a volume reduction, a low reaction temperature (<300° C.) and high pressure (˜20-50 bar) are in favor of the equilibrium conversion. On the other hand, a higher temperature can improve the reaction kinetics. As a result, the catalyst operating temperature window is generally very narrow. Although the widely studied Cu-based methanol synthesis catalysts, such as CuO/ZnO/ZrO₂, do exhibit excellent activities at around 240-260° C., these catalysts suffer from rapid deactivation at temperatures above 300° C. due to Cu sintering. Pelletized catalysts in packed-bed reactors typically encounter poor heat transfer and hot spot formation on the catalysts; therefore, micro-channel reactors, monolithic reactors, and highly conductive structed catalytic reactors have been explored. These designs demonstrated remarkably improved on-stream durability but have not attracted enough attention mainly because of the relatively low space time yield of these systems compared to a packed-bed reactor design. Therefore, proving out the concepts in a large-scale reactor remains a major challenge.

Further, to maximize the synergistic effects it is desirable to place the metal oxides and the acid components in close proximity. When these two components are in close contact with each other, however, detrimental interactions may occur, which hurts the long-term durability of the catalysts. For example, it has been reported that a bifunctional catalyst with CuO/ZnO/ZrO₂ intimately mixed with a Ferrierite zeolite exhibited superior activity for the direct conversion of CO₂ to DME, but also noted that the catalyst progressively lost its CO₂ hydrogenation activity as well as its selectivity to DME in a 150-hour durability test. Migration of Cu to the zeolite component was identified as one of main deactivation mechanisms. To circumvent this issue, bifunctional catalysts with a core-shell structure were explored. Typically, the acid component is placed on the shell that covers the metal oxide core. Thus, methanol formed in the core must diffuse through the shell, where it is dehydrated to DME. Because the interaction of the two components is limited to the interfacial region, core-shell catalysts did show better on-stream durability than the conventional catalysts with the two components simply mixed with each other; however, deactivation is still noticeable. Also, preparation of catalysts with a core-shell structure and good mechanical strength is more than trivial. It typically requires additional binding materials and dense packing of the components, which often reduces the gas diffusivity to the catalytic components. As a result, both experimental results and model simulation have shown that catalysts with a core-shell structure are generally less effective. Therefore, a need exists for a multifunctional catalyst that has at least one of improved durability and gas diffusivity.

### SUMMARY OF THE INVENTION

A multifunctional monolith catalyst is provided. The multifunctional monolith catalyst overcomes the challenges described above and has superior durability, gas diffusivity, heat transfer and/or reaction yields. The multifunctional monolith catalyst includes a monolithic substrate. A first layer is coated on a surface of the substrate. The first layer includes a first catalyst. A second layer is formed on top of the first layer. The second layer includes a second catalyst, and the second layer is porous. Layering of the first and second catalysts reduces degradation of one or both of the first and second catalysts.

In specific embodiments, the substrate is formed of a metal or a fiber material.

In specific embodiments, the substrate is one of a sheet, a corrugated sheet, or a honeycomb structure.

In specific embodiments, the substrate is heat conductive.

In particular embodiments, the substrate includes SiC.

In specific embodiments, the first and second layers each have a thickness in the range of 20 to 300 μm.

In specific embodiments, the first catalyst is a CuZnZr-based catalyst.

In specific embodiments, the second catalyst includes an acid component, such as a zeolite.

In particular embodiments, the second catalyst is a Ferrierite (FER) zeolite.

In specific embodiments, the multifunctional monolith catalyst further includes an inert layer interposed between the first layer and the second layer.

In specific embodiments, the first catalyst is different than the second catalyst, the first catalyst catalyzes a first reaction, and the second catalyst catalyzes a second reaction different from the first reaction.

In particular embodiments, the first catalyst catalyzes the first reaction to form a reaction product, and the second catalyst catalyzes the second reaction in which the reaction product of the first reaction is a reactant.

A method of converting carbon dioxide to dimethyl ether utilizing the multifunctional monolith catalyst is also provided. The method includes providing the multifunctional monolith catalyst in any of the embodiments described above. The method further includes introducing carbon dioxide to the multifunctional monolith catalyst. The carbon dioxide passes through the second layer to the first catalyst of the first layer. At the first layer, the first catalyst catalyzes a reaction of the carbon dioxide to form methanol. The methanol then passes to the second layer in which the second catalyst catalyzes a reaction of the methanol to obtain dimethyl ether. Layering of the first and second catalysts increases a yield of the reaction catalyzed by the second catalyst and increases the longevity of the catalysts.

In specific embodiments, the method is conducted at a temperature in the range of 200° C. to 320° C.

In specific embodiments, the method is conducted at a pressure in the range of 20 to 50 bar.

These and other features of the invention will be more fully understood and appreciated by reference to the description of the embodiments and the drawings.

### BRIEF DESCRIPTION OF THE DRAWINGS

FIG. 1 is a pictorial view of a monolith substrate of a multifunctional monolith catalyst in accordance with embodiments of the disclosure;

FIG. 2 is a schematic view of a multifunctional monolith catalysts in accordance with embodiments of the disclosure;

FIG. 3 is another schematic view of a multifunctional monolith catalysts in accordance with embodiments of the disclosure;

FIG. 4 is a graph of CO₂ conversion and product yields for CO, CH₃OH, DME, and oxygenates (CH₃OH plus DME) for various catalyst configurations;

FIG. 5 is a graph of various bifunctional (CuZnZr+FER) catalysts with different catalyst component proximity;

FIG. 6 is a graph of the catalytic performance of CuZnZr single component catalysts either as pellets (250-500 μm) in a packed-bed reactor or as coated monoliths;

FIG. 7 is a graph of the catalytic performance of bifunctional ((CuZnZr mixed with FER) and (CuZnZr on FER)) catalysts either as pellets (250-500 μm) in a packed-bed reactor or as coated monoliths;

FIG. 8 is a graph of the effect of gas hour space velocity (GHSV) on the catalytic performance of the layered structure bifunctional monolith catalyst;

FIG. 9 is a graph of the on-stream durability of the layered structure bifunctional monolith catalyst; and

FIG. 10 is a graph of kinetic data analysis of the activity data shown in FIG. 8.

### DETAILED DESCRIPTION OF THE CURRENT EMBODIMENTS

As discussed herein, the current embodiments relate to a layered-structure, multifunctional monolith catalyst and a method of converting carbon dioxide to dimethyl ether using the catalyst. The multifunctional monolith catalyst generally includes a monolithic substrate and a plurality of catalysts layered one on top of the other on the substrate. The multifunctional monolith catalyst has at least one of superior durability, gas diffusivity, heat transfer and/or reaction yields. More particularly, the multifunctional monolith catalyst overcomes the major challenge of catalyst deactivation caused by metal sintering and detrimental interactions among the catalytic components of a multifunctional catalyst. The multifunctional monolith catalyst minimizes the undesirable interaction between the catalyst components and drastically improves the on-stream durability of the catalyst, i.e. no activity decline was observed in a 146-hour performance test. In addition, the present layered configuration surprisingly significantly improves the synergistic effects of the two components, resulting in a 20% increase in the productivity for dimethyl ether at 240° C. as compared to conventional bifunctional catalysts. The multifunctional monolith catalyst thereby enables improved direct conversion of carbon dioxide to dimethyl ether by reducing the capital and operating costs of the process and in turn improves energy utilization efficiency.

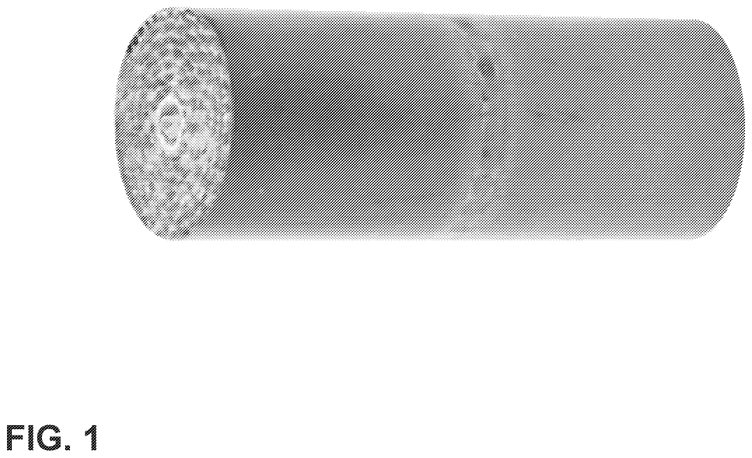

The substrate of the multifunctional monolith catalyst serves as both a support for the catalyst coatings and as a heat conductive medium to regulate the catalyst bed temperature. The substrate may be formed, for example, of a metal or fiber material that has heat conductive properties. In some embodiments, the substrate is formed of or includes an FeCr alloy. In some embodiments, the substrate includes SiC to improve the conductivity. The substrate may be in the form of a simple sheet, for example a foil. In other embodiments, the substrate may be in the form of a corrugated sheet. In certain embodiments, the corrugated sheet may have a generally planar top and bottom layer that sandwich a corrugated layer therebetween. In yet other embodiments, the substrate may have a honeycomb or other similar lattice pattern. In some embodiments, the substrate may be rolled into a cylindrical shape. An example of a suitable substrate is shown in FIG. 1 having a honeycomb, corrugated structure.

A first layer is coated (e.g., washcoated) directly on the external surfaces of the substrate, including channel surfaces of the substrate through which a fluid (e.g., carbon dioxide gas) may flow. The first layer includes a first catalyst capable of catalyzing a first reaction. In particular, the first catalyst may be a methanol synthesis catalyst (capable of catalyzing the formation of methanol from carbon dioxide, i.e. (R1) and/or (R2)) such as a CuZnZr-based catalyst, for example CuO/ZnO/ZrO₂. The direct contact between first catalyst and the substrate allows for excellent heat transfer between the substrate and first catalyst and thereby eliminates hot spot formation that can cause catalyst deactivation. The first layer is generally a thin layer of the first catalyst material, and may have a thickness in the range of 20 to 300 μm, alternatively in the range of 40 to 200 μm, alternatively in the range of 60 to 150 μm, alternatively in the range of 20 to 40 μm, alternatively in the range of 20 to 60 μm, alternatively in the range of 20 to 90 μm, alternatively in the range of 30 to 90 μm, alternatively in the range of 40 to 90 μm.

A separate second layer is coated (e.g., washcoated) over the first layer. The second layer includes a second catalyst capable of catalyzing a second reaction that is different than the first reaction, and the second catalyst includes an acid component. In particular, the second catalyst may catalyze a dehydration reaction (R3) the synthesis of dimethyl ether from methanol (which is the reaction product of the first reaction), and may be a zeolite such as, for example, a Ferrierite (FER) zeolite. The Ferrierite zeolite may have a Si:Al atomic ratio of 10:1. The second layer is generally a thin layer of the second catalyst material, and may have a thickness in the range of 10 to 300 μm, alternatively in the range of 20 to 200 μm, alternatively in the range of 20 to 150 μm, alternatively in the range of 10 to 40 μm, alternatively in the range of 20 to 60 μm, alternatively in the range of 20 to 90 μm, alternatively in the range of 30 to 90 μm, alternatively in the range of 40 to 90 μm. The second layer is porous, such that fluids may flow from the exterior of the multifunctional monolith catalyst through the second layer to the first layer, and from the first layer through the second layer to the exterior. Moreover, in particular embodiments the layered structure of the multifunctional monolith catalyst ensures that carbon dioxide can diffuse through the second layer to the bottom, first layer, and that methanol formed in the bottom, first layer can diffuse through the second catalyst of the second layer before it can exit the multifunctional monolith catalyst. The layered structure of the multifunctional monolith catalyst also limits the direct contact of the metal oxides (first catalyst) and the zeolite components (second catalyst) to the interfacial region and minimizes the potential detrimental interaction between the two components. Different from core-shell designs, the present catalyst coatings on the monolith substrate can be made highly porous if the coatings are adequately adhered to the substrate because it is mainly the substrate that determines the mechanical strength of the element rather than the catalyst coating layers. Also, the catalyst coating thickness for each component can be adjusted independently and over a much wider range compared to core-shell designs. These features enhance catalyst utilization efficiency while offering flexibility to adjust product distribution.

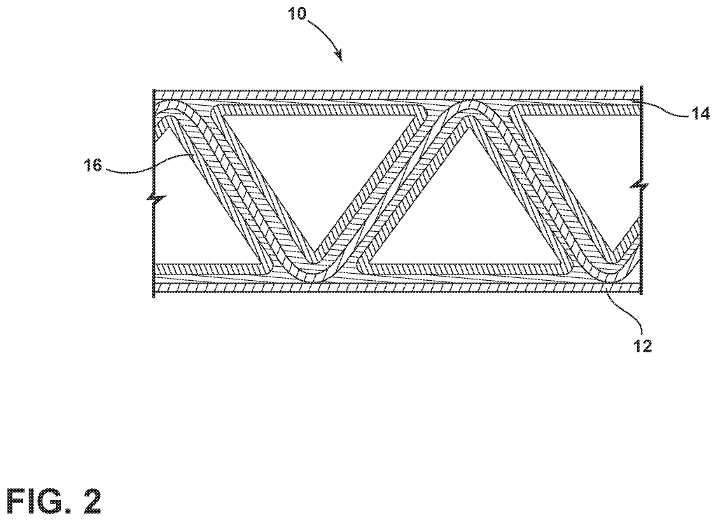

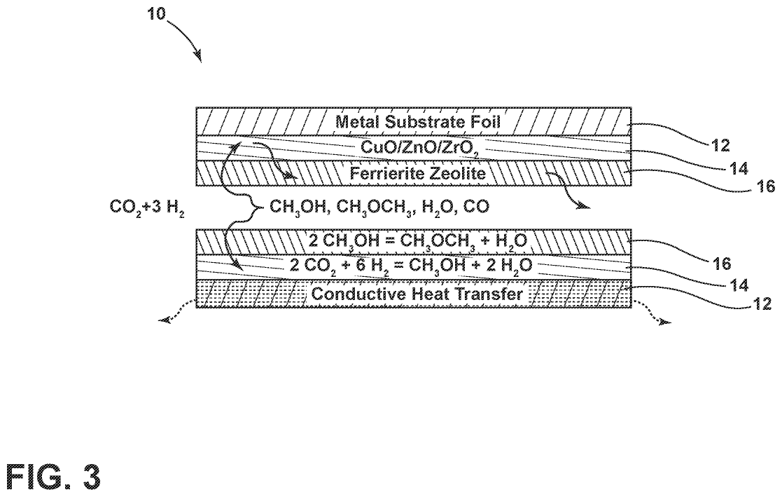

A schematic view of an exemplary embodiment of a layered, multifunctional monolith catalyst 10 is shown in FIG. 2. The substrate 12 is a metallic monolith substrate such as the shown on the right-hand side of FIG. 1. The substrate 12 is formed of layers of metal foils having corrugated foils between the layers. The first layer 14 is formed directly on the substrate 12, and may be, for example, a CuZnZr-type catalyst. The second layer 16 is formed on top of the first layer 14, and may be, for example, a zeolite catalyst. Additional layers, not shown, may be formed on top of the second layer 16 or between the layers 14, 16. Another schematic view of an exemplary layered, multifunctional monolith catalyst in accordance with the disclosure is shown in FIG. 3. The upper portion of the schematic shows the materials of construction of the substrate 12, first layer 14, and second layer 16, while the bottom portion of the schematic shows the function and reactions that take place in each layer. Movement of chemical compounds through the layers 14, 16 is shown in both portions by arrows. Initial reactants are shown on the left, and reaction products are shown in the middle.

In other embodiments, an inert layer of material(s) may be applied between the first and second layers to eliminate any undesirable interaction between the catalyst of the first layer and the catalyst of the second layer.

The catalytic activity and selectivity of the multifunctional monolith catalyst in accordance with embodiments of the disclosure was compared against conventional pelletized bifunctional catalysts in a packed-bed reactor. The results clearly demonstrated the performance advantages of the present layered multifunctional monolith catalyst. More remarkably, a 146-hour on-stream durability test demonstrated excellent stability of the present layered multifunctional monolith catalyst. The following laboratory example are intended to be non-limiting.

CuO/ZnO/ZrO₂ (CuZnZr) catalyst with a mole ratio of (6:3:1) was prepared by a gel-oxalate coprecipitation method. Nitrate salts of Cu, Zn and Zr were dissolved in ethanol to form a 0.1 M mixture solution. A separately prepared oxalic acid solution in ethanol (1 M) was added dropwise to the above metal mixture solution under vigorous stirring until the quantity of oxalic acid reached 120% of the stoichiometric amount of the total metal ions. After mixing overnight, the precipitate was filtered, dried at 110° C., then calcined at 360° C. for 4 hours. The powder sample was either used directly as a CuZnZr component to prepare other CuZnZr containing catalysts or pelletized and sieved to 250-500 μm particles as catalysts.

NH⁺-form Ferrierite zeolite with a Si/Al ratio equal to 10 and a prime crystal size less than 1 μm was obtained from Zeolyst International. The sample was calcined at 550° C. for 4 hours to H⁺-form Ferrierite (FER).

A conventional bifunctional (CuZnZr on FER) catalyst was prepared in a similar way as the CuZnZr catalyst, except that an equal amount of H⁺-form FER was added into the CuZnZr mixture solution before precipitation. A second conventional bifunctional catalyst (CuZnZr+FER, <10 μm) was prepared by mixing equal amounts of the CuZnZr and FER powders by mortar and pestle for 30 minutes to achieve a uniform powder mixture. Both the (CuZnZr on FER) and the (CuZnZr+FER, <10 μm) powders were pelletized and sieved to 250-500 μm particles. A third conventional (CuZnZr+FER, >250 μm) catalyst was prepared by physically mixing the separately pelletized CuZnZr and FER particles (both in the range of 250500 μm) together.

FeCr-alloy metallic monolith substrates with a dimension of 19 mm (diameter)×51 mm (length), 600 cells per square inch (CPSI), and 0.05 mm wall thickness were purchased from Performance Industries. CuZnZr monolith catalysts were prepared by blending the CuZnZr catalyst power with 5% ZrO₂ (added as zirconium acetate) to form a consistent slurry, which was subsequently washcoated onto the metal monolith substrates. The layer structured (CuZnZr II FER) present multifunctional (e.g., bifunctional) monolith catalyst was prepared by applying a second layer of slurry made of FER and 5% ZrO₂ onto the CuZnZr monolith catalysts prepared above. The target catalyst loadings were 61 g (CuZnZr+ZrO₂)·L⁻¹ and 61 g (FER+ZrO₂)·L⁻¹, with respect to the substrate volume. The multifunctional monolith catalysts were dried and calcined at 360° C. for 4 hours.

As discussed below, catalytic activity evaluation was carried out on a flow reactor under a pressure of 25 bar with a feed gas mixture of CO₂:H₂:N₂ equal to 3:9:1 (in moles). The gas flow rates were controlled by corresponding mass flow controllers. For pelletized catalysts, 250-500 μm catalyst particles were first mixed with SiC grits with a particle size ˜365 μm at a weight ratio of 1:5. The mixture was loaded into a packed-bed quartz tube which was inserted into the flow reactor tube. For the present multifunctional monolith catalysts, samples were loaded directly into the flow reactor tube. Gas products were analyzed by an on-line GC (SRI Instruments, Multiple Gas #5) and a gas phase Fourier transform infrared (FTIR) spectrometer (MKS Instruments, Model 2030HS). The GC was used to identify products and the gas phase FTIR was used to quantify CO, CH₃OH, and DME. The product yield was calculated based on the mole of carbons in the product. Hence, 1 mole of DME molecules were counted as 2 moles of C1 product, which made it easier to verify mass balance. With the catalysts and evaluation conditions used herein, CO, CH₃OH and DME were the only detectable C-containing products besides CO₂ which was in the feed gas.

A series of catalysts with the two types of components being placed in various ways were prepared. Details of the different catalyst configurations are listed in Table 1 below. More particularly, CuZnZr-based catalysts were prepared by an oxalate gel coprecipitation method. Zeolites—Ferrierite (FER, with an SiO₂/Al₂O₃=20), ZMS-5 (MFI, with an SiO₂/Al₂O₃=30), and Beta (BEA, with an SiO₂/Al₂O₃=25) were obtained from Zeolyst International. These catalysts were evaluated for the direct conversion of CO₂+H₂ to dimethyl ether (DME) with the formation of methanol (CH₃OH) as an intermediate. The space time yields for the total amount of CH₃OH+DME (as C1) and for each of the two individual products of these catalysts were used to compare their catalytic activities. The values are also listed in Table 1 below.

Comparative Example 1 was 250-500 μm pellets of CuZnZr catalyst (Comp. 1), Methanol was the only HC product over the catalyst. Under the reaction conditions, the catalyst produced 8.74 mmol methanol/g catalyst/h.

Example 1 was a monolith supported CuZnZr catalyst (single catalyst, unifunctional). Under the same reaction conditions as in Comparative Example 1, the single component monolith catalyst produced nearly the same amount of methanol as the catalyst in its pellet form.

In Comparative Example 2, a pelletized ferrierite catalyst was added to the same reactor as in Comparative Example 1 but was placed behind the CuZnZr catalyst as a separate bed. The zeolite catalyst converted about 70% of the methanol formed on the CuZnZr catalyst into DME, with the space time yield for the total CH₃OH+DME remained the same.

Comparative Examples 3-5 were a set of pelletized bifunctional catalysts with the CuZnZr and ferrierite zeolite components being mixed in different ways. Comparative Example 3 was a physical mixture of the two components, each was pelletized to 250-500 μm sizes. In Comparative Example 4, the two components were first mixed as <1 μm powders. The mixture was subsequently pelletized to 250-500 μm sizes. Comparative Example 5 was prepared by coprecipitating CuZnZr onto the surfaces of the ferrierite powders, which was subsequently pelletized to 250-500 μm sizes. These bifunctional catalysts not only converted a significant portion of methanol formed on the CuZnZr component to DME, but also enhanced the overall production of (CH₃OH+DME). The space time yields for (CH₃OH+DME) over the bifunctional catalysts were about 30% higher compared to that for the CuZnZr catalysts. Among the three bifunctional catalysts with different configurations, the promotion effects were largely the same.

Example 2 was prepared according to embodiments of the present disclosure. A ferrierite catalyst layer was coated on top of a monolith supported CuZnZr catalyst as prepared in Example 1. Compared to the various pelletized bifunctional catalysts, surprisingly, the layered-structure bifunctional catalyst exhibited a further increase (by about 22%) of the space time yields for (CH₃OH+DME).

Comparative Example 6 was similar to Comparative Example 3, but an MFI zeolite was used in place of the ferrierite zeolite. Similarly, enhancement in the space time yield for (CH₃OH+DME) was observed for this pelletized bifunctional catalyst as compared to the CuZnZr catalyst (Comp. 1).

Example 3 was a layer-structured bifunctional monolith catalyst in accordance with embodiments of the disclosure, with the same CuZnZr and MFI components as in Comparative Example 6. The layered catalyst configuration further improved the space time yield for (CH₃OH+DME) by about 34% in comparison to the pelletized bifunctional catalyst (Comp. 6).

Example 4 was another layered-structure bifunctional catalyst in accordance with embodiments of the disclosure, with a CuZnZr component as the first catalyst coating and a Beta zeolite as the second catalyst coating. As in Example 2, enhancement of the space time yield for CH₃OH+DME was observed as compared to the CuZnZr catalyst (Comp. 1).

TABLE 1
List of catalysts with different configurations and their catalytic activity
for the direct conversion of CO₂ + H₂ to DME

| | | Space Time Yield (mmol C1/g Cu cat./h) | | |
|---|---|---|---|---|
| Example # | Catalyst Configuration | CH₃OH | DME (as C1) | CH₃OH + DME |
| Comp. 1 | CuZnZr 250-500 μm pellets | 8.74 | 0 | 8.74 |
| Example 1 | CuZnZr washcoated on metal monolith | 9.10 | 0 | 9.10 |
| Comp. 2 | CuZnZr 250-500 μm pellets followed by FER | 2.62 | 6.23 | 8.85 |
| | 250-500 μm pellets in two separate beds | | | |
| Comp. 3 | CuZnZr 250-500 μm pellets mixed with FER | 3.23 | 8.70 | 11.93 |
| | 250-500 μm pellets | | | |
| Comp. 4 | CuZnZr mixed with FER as <1 μm powders, | 2.91 | 8.07 | 10.98 |
| | then pelletized to 250-500 μm | | | |
| Comp. 5 | CuZnZr coprecipitated onto FER, then | 3.16 | 8.28 | 11.45 |
| | pelletized to 250-500 μm | | | |
| Example 2 | CuZnZr first layer, FER second layer | 3.79 | 10.22 | 14.02 |
| | washcoated on metal monolith | | | |
| Comp. 6 | CuZnZr 250-500 μm pellets mixed with MFI | 2.40 | 7.43 | 9.84 |
| | 250-500 μm pellets | | | |
| Example 3 | CuZnZr first layer, MFI second layer | 3.47 | 9.75 | 13.22 |
| | washcoated on metal monolith | | | |
| Example 4 | CuZnZr first layer, BEA second layer | 3.66 | 5.98 | 9.64 |
| | washcoated on metal monolith | | | |

Further, the catalytic performance of the present multifunctional monolith catalyst and a conventional bifunctional catalyst as pelletized particles in a packed-bed reactor were assessed for the direct conversion of CO₂ to DME. CuO/ZnO/ZrO₂ (CuZnZr) as described above was selected as the methanol synthesis catalytic component as it has exhibited better activity and stability than the commercial CuO/ZnO/Al₂O₃ catalyst for methanol synthesis (R2) with CO₂ as feedstock. For methanol dehydration to DME, a Ferrierite (FER) zeolite with a Si/Al atomic ratio of 10 was chosen as described above. Zeolites, owing to their hydrophobicity, are more suitable than the more commonly studied γ-Al₂O₃ as a solid acid for the application of CO₂ as the feedstock because H₂O is generated in both steps and zeolites are more tolerant to high concentrations of H₂O than γ-Al₂O₃ for the dehydration reaction (R3). Ferrierite zeolites with Si/Al ratios around 10 exhibit high activity and on-stream stability because of their unique 10-membered ring framework structure and balanced acidity and hydrophobicity properties. Conventional bifunctional catalysts with nanoparticles of CuZnZr directly deposited on the external surfaces of ˜1 μm FER to achieve intimate contact between the two components (CuZnZr on FER) were also prepared as described above.

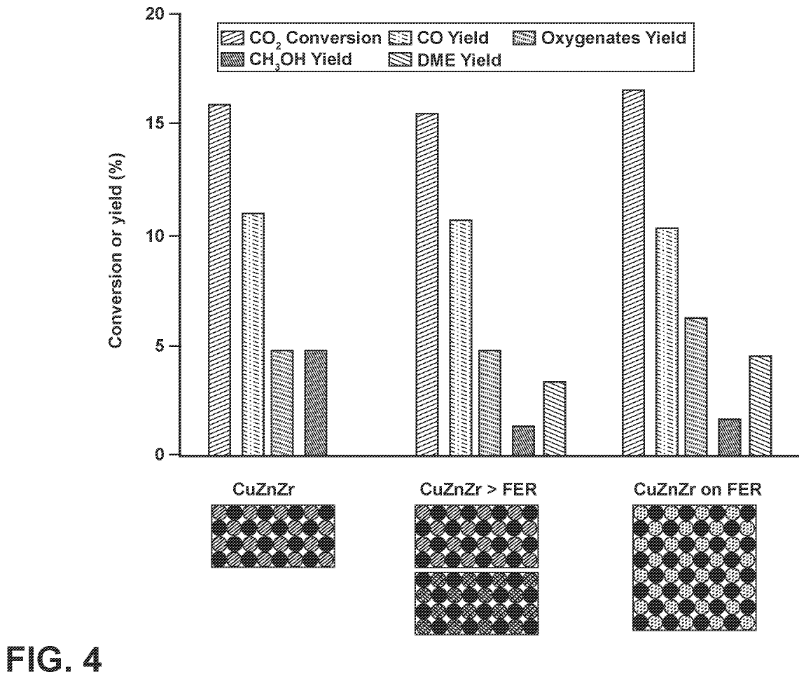

FIG. 4 summarizes the CO₂ conversion and the product yields for CO, CH₃OH, dimethyl ether (DME), and oxygenates (CH₃OH+DME) over the pelletized catalysts (described in the preceding paragraph) in a fixed-bed reactor at 240° C. and 25 bar with a feed of CO₂:H₂:N₂ equal to 3:9:1 (in moles) and a flow rate of 15.6 L (STP) per hour. The catalyst loadings were 0.883 g each of CuZnZr and FER (when it was included). For these tests, SiC grits (˜365 μm) as a diluent was physically mixed with the catalyst pellets (250-500 μm) at a weight ratio of 5:1 to improve heat transfer. At 240° C., all the catalysts reached the peak conversions for oxygenates. CH₃OH, DME, and CO were the only detectable products. By itself, CuZnZr catalyst (left-hand side of the graph) achieves 15.9% CO₂ conversion, with CH₃OH as the only oxygenate HC product at a yield of 4.8%. Simply placing an equal amount of the FER component downstream of the CuZnZr catalyst as two separate beds (CuZnZr>FER; middle portion of graph) has no effect on CO₂ conversion or the yield of total oxygenates. What the FER catalyst does is simply convert about 70% of the methanol produced by the upstream CuZnZr catalyst to DME, resulting in a simple additive effect. In contrast, the bifunctional catalyst (CuZnZr on FER; right-hand side of graph) with the two components in close contact shows an increase of CO₂ conversion to 16.6%; the total mass in this evaluation was equivalent to the CuZnZr>FER evaluation. The yield for the total oxygenates (counted as C1) increased to 6.3% and the selectivity for DME also increased slightly (to 73%). These results reaffirm the advantages of the bifunctional catalysts for the direct conversion of CO₂ to DME.

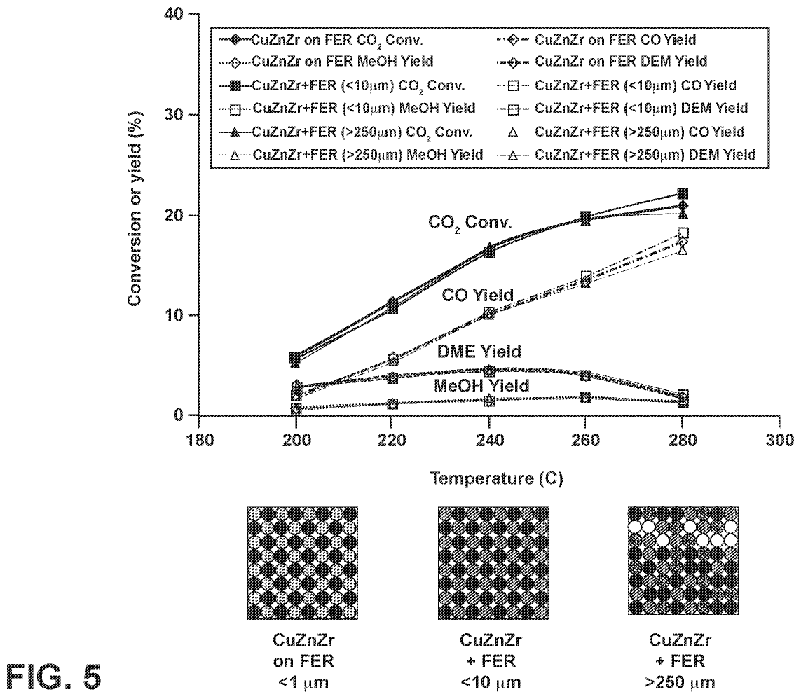

Further, to assess if the proximity of the CuZnZr and FER components affects the performance of the bifunctional catalysts, two additional catalysts with identical compositions as the (CuZnZr on FER) catalyst were prepared by gradually increasing the grain sizes of the individual component. A (CuZnZr+FER, <10 μm) catalyst was prepared by mixing a separately prepared CuZnZr powder sample with the FER zeolite by mortar and pestle to form a uniform powder mixture before it was pelletized into 250-500 μm particles. It is estimated the grain sizes of each individual component for this catalyst are less than 10 μm. Another catalyst (CuZnZr+FER, >250 μm) was a physical mixture of CuZnZr and FER pellets, each being pelletized separately to a particle size of 250-500 μm. FIG. 5 compares the CO₂ conversion and the product yields of CO, CH₃OH, and DME in the temperature region of 200 to 280° C. for the three catalysts (CuZnZr on FER, CuZnZr+FER, <10 μm, and CuZnZr+FER, >250 μm). All show almost identical performance. The results demonstrate that, with the two components used herein, mixing the two components with individual grain sizes in the range of ˜1 to 500 pin can achieve the same level of synergistic effects as directly depositing CuZnZr nanoparticles onto FER zeolite. This suggests that, with this system, gas phase methanol is the only intermediate and the direct conversion of CO₂ to DME proceeds through (R2) and (R3) in tandem. Otherwise, if other surface intermediates are involved in the reactions, one would expect the performance of the catalysts to change with the modification of the proximity of the two components.

Coating the CuZnZr and FER catalysts on monolith substrates does not alter the fundamental catalytic properties of the components but does affect how the two components interact with each other. CO, CH₃OH, and DME (when the FER catalyst is present) remain to be the only detectable products. For clarity and a fair composition, we use productivity, defined as millimoles of oxygenate products (counted as C1) formed per gram of the CuZnZr component in the catalyst at a given time (per hour), to compare the catalytic performance of the catalysts tested either as pellets in a packed-bed reactor or as monolith flow reactors.

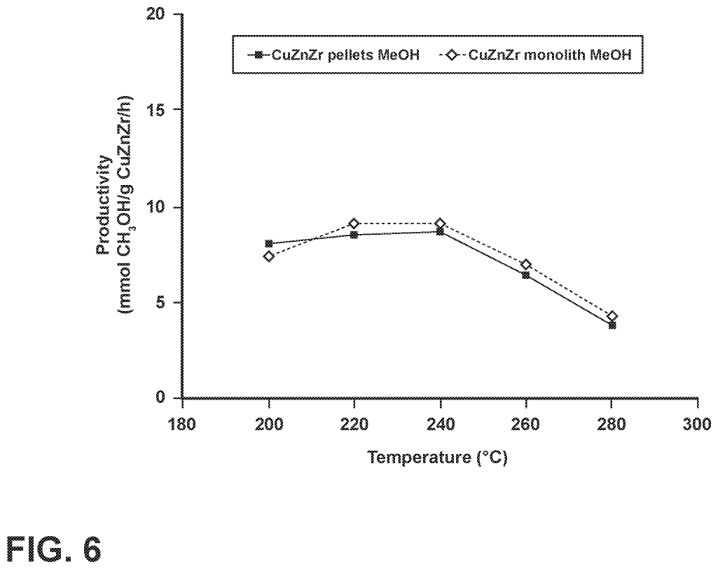

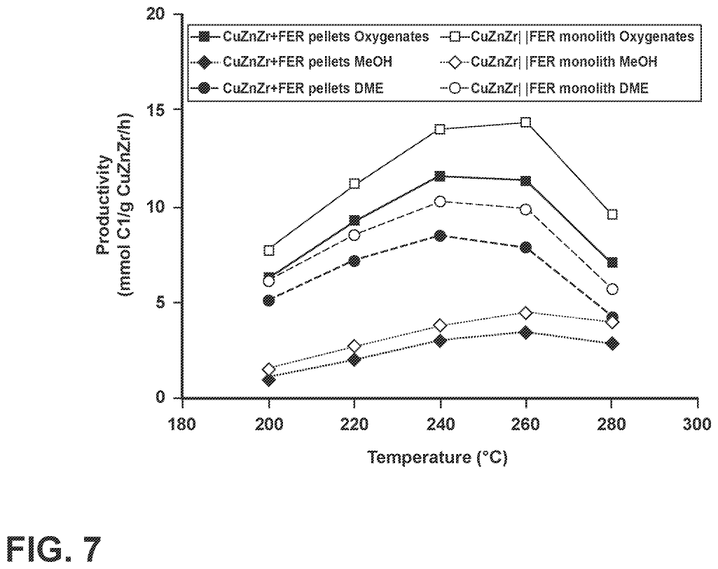

FIG. 6 compares the productivity of methanol as a function of the catalyst inlet temperature for the pelletized and the metal monolith supported CuZnZr catalyst. The reactions were carried out at 25 bar with a feed of CO₂:H₂:N₂ equal to 3:9:1 (in moles) and a flow rate of 15.6 L (STP) per hour. The catalyst loadings were 0.883 g each of CuZnZr. Both sets of results are nearly identical. This is not surprising and confirms that the present coating process does not alter the catalytic function of the CuZnZr component. On the other hand, surprisingly significant improvements in productivities for DME and CH₃OH (and for the total oxygenates) are observed for the present layer structured (CuZnZr∥FER) monolith catalyst with the CuZnZr component in the bottom layer and the FER component in the top layer as compared to a conventional bifunctional catalyst in which the two components are mixed as pellets. The results are shown in FIG. 7. At 240° C., where DME reaches its peak conversions for both catalysts, the DME productivity increased by 20% from 8.5 to 10.2 (mmol C1·g⁻¹ CuZnZr·h⁻¹). The productivity for CH₃OH also increases by a similar percentage from 3.0 to 3.8 (mmol C1·g⁻¹ CuZnZr·h⁻¹), or 27%. As demonstrated by the results in FIG. 5, mixing the CuZnZr and FER components in different levels of proximity all achieve the same level of synergetic effects, the additional performance enhancement observed in FIG. 7 for the present monolith catalyst is clearly due to the layer structured configuration although more uniform temperature distribution within the metallic monolith catalyst may also contribute to the improvement.

One general concern in using monolith catalysts for industrial chemical process applications is that the catalysts may require a large reactor space because of the void spaces in the monolith channels, which results in a low space time yield of the reactor. While space is even more constrained in automotive applications, monolith catalysts are installed on every modern vehicle to abate the toxic pollutants from the engine exhaust. For automotive applications, the open channels of the monolith catalysts are actually beneficial in reducing the pressure drop in the exhaust pipe. The presence of a large amount of void space for a monolith catalyst is compensated by more effective catalyst utilization because the thin coatings allow more feed gas to flow through the channels and still achieve efficient high conversions.

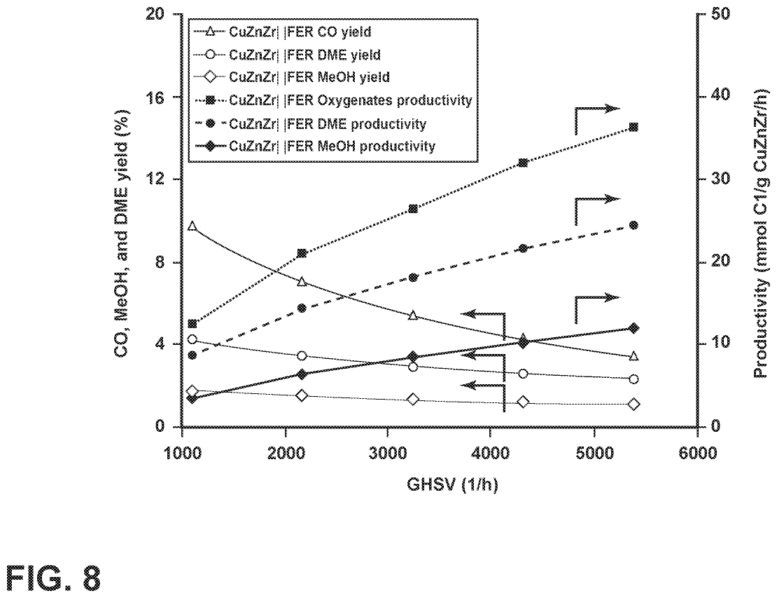

To probe how the present bifunctional monolith catalyst responds to the increase of feed gas flux, the gas flow rates were gradually increased from 15.6 L·h⁻¹ to 78.0 L·h⁻¹ while the volume of the monolith core sample was kept the same. Accordingly, the gas hour space velocity (GHSV) over the monolith catalyst was increased from 1078 to 5390 h⁻¹. The reactions were carried out at 25 bar with a feed of CO₂:H₂:N₂ equal to 3:9:1 (in moles). The results are shown in FIG. 8, which indicate that although the yields of CO, CH₃OH, and DME decrease with the increase of gas flow rates, the reduction is not proportional but at much gradual slopes. As a net result, the productivities for the products of DME and CH₃OH at a given amount (volume) of catalyst increase noticeably. For example, the DME productivity increases by a factor of about 3 from 8.7 to 24.3 (mmol C1·g⁻¹ CuZnZr·h⁻¹) by increasing the feed gas flow rate by a factor of 5. It is worth noting that the reduction in CO yield is more pronounced than that for DME and CH₃OH with the increase of gas flow rate. Therefore, increasing the feed gas flux not only improves the productivities for oxygenates but also increases their selectivity. Furthermore, as the layered-structure multifunctional monolith catalysts are supported on a monolith substrate such as a honeycomb or corrugated structure, the pressure drop over the present catalysts is generally lower than that over a catalyst bed of pelletized catalyst. This makes it possible to further intensify the process by increasing the feed gas flow rate without causing backpressure concerns.

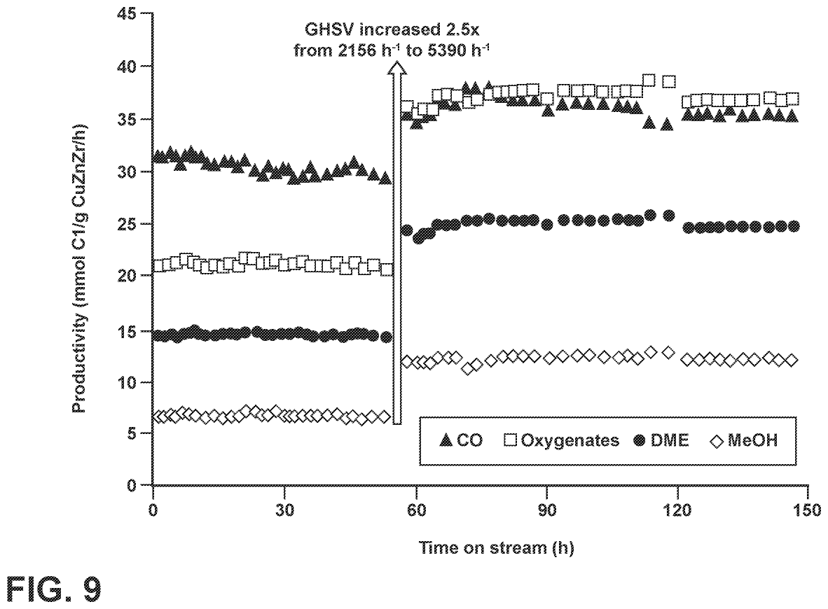

The on-stream durability of the present bifunctional (CuZnZr∥FER) monolith catalyst was also evaluated at 240° C. The reactions were carried out at 25 bar with a feed of CO₂:H₂:N₂ equal to 3:9:1 (in moles). In the first 53 hours of the activity testing, the GHSV was set at 2156 h⁻¹. Thereafter, it was increased to 5390 h⁻¹. The results are shown in FIG. 9. During the 146 hours of durability testing, the present bifunctional monolith catalyst was remarkably stable, unlike other multifunctional catalysts. No decline in the productivities of DME and CH₃OH was observed. There was a small downward trend in CO formation in the first 25 hours of testing, but it became stable in the rest of the evaluation time. Other small variations for the data points were due to test-to-test variabilities in setting up the testing conditions and the analytic equipment.

The additional enhancement in the productivities for DME and CH₃OH for the present layered structured (CuZnZr II FER) bifunctional monolith catalyst as compared to the conventional pelletized bifunctional catalysts is unexpected. Uniform temperature distribution for the monolith catalyst facilitated by the metallic substrate could have some contributions. However, since a considerable amount of SiC diluent was used in the packed-bed reactor and the diameter of the packed bed reactor is relatively small (ID=16 mm), we believe that the temperature distribution in the packed-bed reactor was reasonably uniform as well although we made no attempt to measure the temperature gradient within the catalyst bed. Also, the data in FIG. 7 show that the performance of the bifunctional catalysts in either a monolithic or packed-bed configuration is relatively insensitive to temperature variation around 240-260° C. This leads us to conclude that it is mainly the layer structured configuration in the present monolith catalyst that contributes to the additional performance improvement.

As illustrated in FIG. 3, with the present layered structure configuration, feed gas of CO₂ and H₂ would first diffuse through the top layer of FER component to reach the bottom CuZnZr catalyst, where they are converted to CH₃OH and H₂O (R2). Because the catalyst coatings are porous, there is no diffusion barrier for CO₂ and H₂ to reach the CuZnZr catalyst. In the subsequent step, the produced CH₃OH (and H₂O) would have to pass through the FER layer before it enters the gas phase in the channels and exits the reactor. Thus, the layered configuration ensures all CH₃OH molecules formed have opportunities to react on the zeolite catalyst. Additionally, for the CH₃OH molecules that do not react and find their way to reach the gas phase in the monolith channel, if these molecules diffuse back to the catalyst, they will have to pass through the zeolite layer again before reaching the CuZnZr layer. This provides a second opportunity for the CH₃OH to be converted to DME. Therefore, it is possible that the CH₃OH concentration in bottom CuZnZr layer of the monolith catalyst is lower than that for the pelletized catalyst in which the CuZnZr component is exposed directly to the gas phase. A lower concentration of CH₃OH on the surface of the CuZnZr component drives (R2) forward.

The notion that the product yield for the direct conversion of CO₂ to DME over the present monolith catalyst is largely controlled by the thermodynamic equilibrium of the chemical reactions (R2), (R3), and (R4) is supported by an empirical kinetic data analysis of the data shown in FIG. 8. For this purpose, (R3) will be re-written as:

CO₂+3H₂=½(CH₃—O—CH₃)+3/2H₂O (R3′)

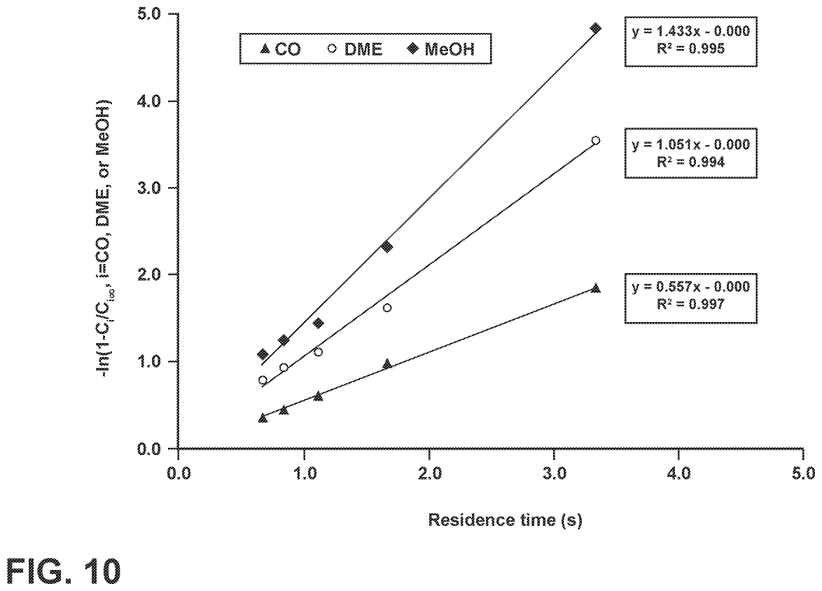

The reason of introducing ½(CH₃—O—CH₃) is that in our data analysis we count 1 mole of DME as 2 moles of C1 product. Therefore, for (R3′) we consider ½(CH₃—O—CH₃) as a pseudo-molecule. By treating (R2), (R3′), and (R4) as three independent reactions that follow the first order of kinetics near their equilibrium points with respect to the concentrations of CO₂ and the product of CO, CH₃OH or DME, we can reprocess the data in FIG. 8 as a function of −ln(1−Ci/Ci∞) versus the residence time (the inverse of GHSV), where: i is CO, DME (as C1), or CH₃OH; Ci represents the concentration of each product at a given residence time; and Ci∞ represents the concentration of each product at the equilibrium point. The reprocessed data are plotted in FIG. 10. Each set of data points nicely fit into a straight line that intersects at the origin with a R²>0.99. This reaffirms that all three reactions (R2, R3, and R4) are near their thermodynamic equilibrium points.

Because the product yield is largely controlled by the thermodynamic equilibrium, carrying out the reaction at a higher space velocity (or a shorter residence time) to stay away from the equilibrium point favors productivity as demonstrated in FIG. 8. Under our reaction conditions (25 bar pressure and 240° C.), the present monolith catalyst achieved a productivity of 12.3 mmol C1 of (DME+CH₃OH)·g⁻¹ CuZnZr·h⁻¹ at a GHSV of 1078 h⁻¹, which is comparable to what has been reported for a conventional bifunctional catalyst under nearly identical conditions except at a higher reaction pressure (30 bar). Increasing the GHSV by a factor of 5 to 539011-1, we increased the oxygenates productivity by a factor ˜3 to 36.3 mmol C1 of (DME+CH₃OH)·g⁻¹ CuZnZr·h⁻¹. This partially compensates for the lower space volume utilization efficiency associated with monolith catalysts but will lead to more gas recirculation in practical applications. More detailed technoeconomic analysis is needed to address the tradeoffs. The other approach to increase the space time yield of monolith catalysts is to increase catalyst loadings at a given volume. As the coating process herein was not optimized, a process optimization could increase the catalyst loading by a factor of ˜2-3.

The most significant advantage of the present layered structure monolith catalyst is the excellent on-stream stability as demonstrated in FIG. 9. No decline in DME or CH₃OH productivity was detected during the 146-h durability test. As a comparison, a steady catalyst deactivation (˜30%) over the course of 100-h evaluation was reported for a conventional bifunctional (CuZnZr on FER) catalyst with nearly identical composition as our (CuZnZr II FER) monolith catalyst. The remarkable results highlight the unique features that monolith substrates can bring into novel catalyst designs for industrial chemical processes: (1) using metallic substrates (or other highly conductive materials, such as SiC monolith substrates) to improve heat transfer which eliminates hot spot formation that can cause catalyst deactivation; and (2) the channel/wall structure enables complex catalyst designs to not only enhance the desirable synergetic effects but also minimize the detrimental interactions among different catalytic components. Another advantage of using the present layered monolith catalyst is that the reactor can be modular in design. The present layered monolith catalyst has wide adaptation with a quick response and robust design, which are all highly desirable for systems that are designed for CO₂ conversion using renewable energy sources as most of the sources are decentralized in remote locations with intermittent surplus capacity.

By way of example, a method of converting carbon dioxide to dimethyl ether using the present multifunctional monolith catalyst includes forming a reactor including the catalyst. Carbon dioxide and hydrogen is introduced to the multifunctional monolith catalyst. The carbon dioxide passes through the second layer to the first catalyst of the second layer, wherein the first catalyst catalyzes a reaction of the carbon dioxide to form methanol (R2). The methanol then passes to the second layer in which the second catalyst catalyzes a reaction of the methanol to obtain dimethyl ether (R3). The method may be conducted at a temperature, for example, in the range of 200° C. to 320° C., and a pressure in the range of 20 to 50 bar.

It is noted that the multifunctional (e.g., bifunctional) layered monolith catalyst is described herein by specific example as being applied to the direct conversion of CO₂ to DME, the layered structure monolith catalyst disclosed herein may be applicable to a wide range of other chemical processes that involve tandem chemical reactions. Thus, by varying the specific catalyst materials incorporated into the layers of the monolith catalyst, the present multifunctional layered monolith catalyst may be used in other applications involving tandem catalytic reactions, such as but not limited to direct conversion of CO₂ to olefins, direct conversion of CO₂ to aromatics, and direct conversion of CO₂ to gasoline. The present multifunctional layered monolith catalyst thereby enhance productivity, improve the energy utilization efficiency, and reduce capital investment and operating costs for such processes.

The above description is that of current embodiments of the invention. Various alterations and changes can be made without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular.

What is claimed is:

1. A multifunctional monolith catalyst comprising:
a monolithic substrate;
a first layer coated on a surface of the substrate, the first layer including a first catalyst;
a second layer formed on top of the first layer, the second layer including a second catalyst, wherein the second layer is porous;
wherein layering of the first and second catalysts reduces degradation of one or both of the first and second catalysts.

2. The multifunctional monolith catalyst of claim 1, wherein the substrate is formed of a metal or a fiber material.

3. The multifunctional monolith catalyst of claim 1, wherein the substrate is one of a sheet, a corrugated sheet, or a honeycomb structure.

4. The multifunctional monolith catalyst of claim 1, wherein the substrate is heat conductive.

5. The multifunctional monolith catalyst of claim 1, wherein the substrate includes SiC.

6. The multifunctional monolith catalyst of claim 1, wherein the first and second layers each has a thickness in the range of 20 to 300 μm.

7. The multifunctional monolith catalyst of claim 1, wherein the first catalyst is a CuZnZr-based catalyst.

8. The multifunctional monolith catalyst of claim 1, wherein the second catalyst includes an acid component, such as a zeolite.

9. The multifunctional monolith catalyst of claim 8, wherein the second catalyst is a Ferrierite (FER) zeolite.

10. The multifunctional monolith catalyst of claim 1, further including an inert layer interposed between the first layer and the second layer.

11. The multifunctional monolith catalyst of claim 1, wherein the first catalyst is different than the second catalyst, the first catalyst catalyzes a first reaction, and the second catalyst catalyzes a second reaction different from the first reaction.

12. The multifunctional monolith catalyst of claim 11, wherein the first catalyst catalyzes the first reaction to form a reaction product, and the second catalyst catalyzes the second reaction in which the reaction product of the first reaction is a reactant.

13. A method of converting carbon dioxide to dimethyl ether utilizing the multifunctional monolith catalyst of claim 1.

14. A method of converting carbon dioxide to dimethyl ether, the method comprising:
providing the multifunctional monolith catalyst of claim 1;
introducing carbon dioxide to the multifunctional monolith catalyst;
wherein carbon dioxide passes through the second layer to the first catalyst of the first layer, wherein the first catalyst catalyzes a reaction of the carbon dioxide to form methanol, the methanol passes to the second layer in which the second catalyst catalyzes a reaction of the methanol to obtain dimethyl ether;
wherein layering of the first and second catalysts increases a yield of the reaction catalyzed by the second catalyst.

15. The method of claim 14, wherein the method is conducted at a temperature in the range of 200° C. to 320° C.

16. The method of claim 14, wherein the method is conducted at a pressure in the range of 20 to 50 bar.
